(19) United States
(12) Patent Application Publication
(10) Pub. No.: US 2006/0178607 A1
(43) Pub. Date: Aug. 10, 2006

(54) Back pressure relief device

(76) Inventor: Alfred Evans, Clarksville, TN (US)

Correspondence Address: NATIONAL IP RIGHTS CENTER, LLC;SCOTT J. FIELDS, ESQ., 550 TOWNSHIP LINE ROAD, SUITE 400, BLUE BELL, PA, 19422, US

(21) Appl. No.: 11/053,296

(22) Filed: Feb. 7, 2005

(51) Int. Cl. A61F 5/00

(52) U.S. Cl. 602/33; 602/32; 602/36

(57) ABSTRACT

A system for relieving back pressure comprising: a waist belt affixed to a reclining individual; a plurality of tension lines extending axial through the legs of the individual; and a mechanism to attach the tension lines so as to provide positive tension.

### FIELD OF THE INVENTION

The present invention is directed to the field of back pressure relief devices. In particular, the present invention is an apparatus, similar to an inversion table, which relieves back pressure through decompression.

### BACKGROUND OF THE INVENTION

The back is one of the most difficult parts of ones body to care for and requires early management. The critical factors in supporting the back are proper exercising of muscle, hamstring, correct posture, and especially minimizing vertabrae compression. The soft vertebral cushion separating the discs can become compressed to the point that nerve protection can be reduced and cushion effect lost.

There have been a number of patents directed to the area of back pressure relief devices. U.S. Pat. No. 5,176,707 discloses a method and apparatus in which a generally vertical force is applied to a person's lower spine in approximately a perpendicular direction to the axis of elongation of the spine. In particular, the person is placed on his back in a substantially supine position. A restraining belt is then placed about the person's pelvic area and a lifting force is applied in an upward direction. The person can then apply pressure to one of various footrests to adjust the amount and direction of the force applied to the spine for maximum relief from lower back discomfort. The method and apparatus may also be used to adjust other body areas, such as the shoulders or neck, by applying the belt to the pertinent body area.

U.S. Pat. No. 4,838,250 discloses an apparatus for applying deep, steady pressure into the deep tissue layers of the muscles of a human user and to “adjust” the vertebrae of the spine, to relieve back pain in a human user. The apparatus is comprised principally of a frame that is collapsible and adjustable, and a back-engaging member, whereby a human user can support himself by resting his arms and hands on the arm-rest members, by resting his feet on the foot-rest members, and be leaning his back against the back-engaging member, and when the user pushes his body weight against said back-engaging member, and if he chooses, takes his feet off the said foot-rest members to allow his feet to be suspended off the ground, the force of gravity as well as slight movement on the part of the user, causes the back-engaging member to apply a deep, steady, slowly sliding pressure into the muscles of the dorsal side of a user's body, relaxing and releasing muscle tension and effecting a spinal adjustment.

U.S. Pat. No. 5,105,804 discloses a method for producing mild, symmetrical (or asymmetrical) traction on lumbar invertebral discs and sacroiliac joints. A patient is supported in water in a spa for approximately 30 minutes by means of an annular floatation device engaging undersides of the patient's arms a predetermined distance from the patient's armpits, the spa being sufficiently deep that the patient's feet cannot touch the bottom. A submerged, symmetrical weight belt is supported on the patient's hips, the buoyant weight of the belt being in the range from approximately 10-20 pounds. The temperature of water in the spa is maintained in the range from approximately 88-94 degrees F. Preferably, the patient remains relatively motionless in the spa for a warm up/relaxation period of approximately 5-10 minutes, followed by a sequence of slow extension and flexion exercise of the patient's back, following by a plurality of side bends.

U.S. Pat. No. 4,603,689 discloses a method and device for self-applying natural traction to the lower spinal region with an elongate generally vertical frame contoured to fit the backrest portion of a furniture piece having a seat, the frame having an upwardly extending portion with a pulley system mounted thereon at a substantial level above the backrest. A cable is trained around the pulley system and a removable belt attached to one end of the cable is adapted to be secured around the lower thoracic region of the user. The other end of the cable is free and is passed through a releasable gripper device mounted on the frame to hang therebelow. The frame is first placed on the backrest of the furniture piece such that the pulley system and free end of the cable overlie the seat. Then a seated position in the chair is taken and the belt is snugly secured around the lower thoracic area of the body. By grasping the free end of the cable and pulling it downwardly, the buttocks are raised vertically such that the weight of the body is substantially wholly supported by the belt and the forces of gravity apply a natural traction to the lower spinal vertebra.

U.S. Pat. No. 5,540,643 discloses an apparatus for creating traction in a user back and is securable to an apparatus support structure having an apparatus engaging rim and an apparatus upright support face includes an elongate suspension member having a first member end and a second member end, a hook structure extending from the first member end in a first lateral direction for engaging the engaging rim, a support arm secured to and extending from the suspension member in a second lateral direction substantially opposite from the first lateral direction to project away from the upright support face, so that downward loading on the arm member causes the suspension member to pivot on the hook structure to abut and find support against the upright support face. A method of performing traction exercises using the above-described apparatus includes the steps of hooking two of the suspension members over an elevated portion of the support structure so that the members are laterally spaced apart from each other, placing the user back against the support structure between the suspension members, raising the user arms over and around the support arms and bending and transferring user weight from the user legs to the support arms, thereby causing the weight of the user torso and user legs to pull downward on the user spine to create traction.

U.S. Patent Application No. 20030130696 discloses a portable spinal adjustment apparatus and method for assisting a user thereof to adjust the user's spine. The apparatus includes an elongate member which defines a substantially convex substantially rigid surface which is disposed between a first end and a second end of the elongate member. The convex rigid surface features a radius of about 2 to 4 inches, thereby defining a first side and a second side, and a length suitable to cooperate with and transversely support a body region of the user. The first end of the elongate member supports the elongate member during use thereof such that the convex rigid surface is facing upward, and the second end of the elongate member also supports the elongate member during use thereof such that the convex rigid surface is horizontal. So disposed and defined by the present invention, the convex rigid surface manifests an arcuate fulcrum, whereby employment thereof combined with the effect of gravity and simple leverage, the user is assisted in adjusting the user's spine.

U.S. Pat. No. 5,458,617 discloses an apparatus for applying traction to an arm or a leg, an elongated body of elastic fabric is provided, which has a plurality of evenly distributed pockets that are elongated transversally of the body. Thin lead strips fit in the pockets as stiffeners. Complementary hook and loop fasteners secure the device around the girth of the arm or leg, so that all of the limb is surrounded by stiffener strips. The inner face of the elastic fabric is provided with a layer of anti-slip material, such as foam rubber, which extends beyond the elastic fabric, both lengthwise and widthwise, of the device. By preference, the device is arcuate in plan and the pockets and stiffener strips correspondingly trapezoidally shaped, when intended to fit around the tapering part of a limb.

U.S. Pat. No. 6,042,602 discloses an apparatus for effecting spinal traction on a patient which includes a tub containing liquid in which a patient, buoyantly supported by a floatation device, can float in a generally upright position in a state of buoyant equilibrium. The level of the liquid in the tub can be varied, which effects a variation on a traction force acting on the patient as the floatation device seeks new positions of buoyant equilibrium in response to the change in liquid level. The traction force may be generated by one or more weighted devices worn on the body of the patient or by a harness attached to the patient and secured within the tub to resist upward movement of the patient when the liquid level rises in the tub. The liquid level within the tub is varied by a liquid level varying apparatus associated with the tub which operates generally by displacing a volume of liquid within the tub to vary the level of the liquid by an amount corresponding to the volume of displaced liquid. A method for effecting spinal traction includes floating a patient in a vessel of water, applying a body-tensioning force to the patient's body, and raising and lowering the level of liquid in the vessel to effect variations in the body-tensioning force as the patient's floating body seeks a new position of buoyant equilibrium.

U.S. Pat. No. 5,290,307, to Choy, discloses an apparatus for an acupressure belt for the treatment of lower back pain is formed of a central fabric panel to which opposed elastic panels are affixed. The elastic panels terminate in a pair of end panels which may be joined together to retain the belt about the torso of the wearer. Located on the inner surface of the central panel is a plurality of acupressure-applying protrusions, each of which are individually positionable on the panel. The protrusions are positioned to apply pressure to the L.sub.1-L.sub.4 acupressure points associated with the lower spine. A pair of overlapping elastic panels are further affixed to the opposed sides of the control panel about the torso with the protrusions in position, whereby acupressure is generated and applied for the relief of lower back pain.

U.S. Patent Application No. 200330105417 discloses a device called PAIN AWAY, which consists of two handgrips on either side of a treatment core. This treatment core has a pair of permanent belts mounted on wheels for pressure treatment of the muscles beside the spine, to relieve back pain and produce relaxation. A detachable belt is used for cellulite treatment. The device vibrates as desired. Since this is the first device based on the acupressure method of back pain relief it is unique in concept. Drake's search of existing patents as provided by the PTDL in Dallas, Tex. #606/204 revealed nothing like PAIN AWAY. Specific features that make it unique are: (1) the fact that it rolls up and down the back; and other parts of the body, eliminating the use of creams or abrasive rubbing motions; (2) the pyramids on the belts need not ever touch the skin itself thereby lessening the chance of transmittal of any infections from one person to another (if the device is used in a commercial manner such as hospital therapy); (3) the fact that the spinal gap spacer (and vibrator housing) (B) makes it ideal for the treatment of spinal pain associated with muscle tension; (4) the fact that the pyramids (S) on the belts (L, M) are rubber will prevent injury: (5) the fact that the simplicity of the use of PAIN AWAY makes it easy for a non-professional to use easily: (6) the fact that it is a cellulite treatment method that does not require lotions and cremes.

While there has been a substantial number of prior art systems, none have facilitate easy application, usage and portability. None have been used with beds and none have been portable.

It is an object of the present invention to provide a back relief system which provides pressure on the back of a user.

It is another object of the present invention to provide a back pressure relief system which can be easily packed and set up by a user in a number of locations.

It is yet a further object of the present invention to provide a back relief system which can be adjusted for the needs of different users.

It is a further object of the present invention to supply a timing clock to prevent too long of a compression of the muscles and discs which can cause muscle inflammation and pain especially if the user accidentally exceeds a reasonable gentle compression period.

These and other objects of the invention will become apparent from the detailed description which follows.

### SUMMARY OF THE PRESENT INVENTION

In accordance with the present invention, a system for relieving back pressure is disclosed. The invention comprises a waist belt affixed to a reclining individual; a plurality of tension lines extending axial through the legs of the individual; and a mechanism to attach the tension lines so as to provide positive tension.

In a further embodiment, this invention is a system for relieving back pressure comprising: a waist belt affixed to a reclining individual; a plurality of expandable tension lines extending axially through the legs of the reclining individual and which terminate with a catch; and an adjustable pole affixed to the catch for adjusting the height of the catch end for providing constant pressure on expandable tension lines.

In still a further embodiment, the invention is a system for relieving back pressure comprising: an adjustable waist belt affixed to a reclining individual; a plurality of expandable tension lines extending axially through the legs of the reclining individual and which end with a catch; and an adjustable pole for affixing the height of the catch end for providing constant pressure on the back of the reclining individual; and a means coupled to the pole for adjusting the tension of the tension lines.

In still a further embodiment, the invention is a method for relieving back pressure comprising the following steps: applying a waist belt to a reclining individual; attaching a plurality of expandable tension lines extending axially through the legs of the user and which end with a catch; and affixing the catch to an adjustable pole for affixing the height of the catch end for providing constant pressure.

### BRIEF DESCRIPTION OF THE FIGURES

FIG. 1 is a perspective view of the back relief system of the present invention.

FIGS. 2 to 6 illustrates the use of the invention showing use by an end user in various positions.

FIG. 7 is an alternative embodiment of the invention.

FIG. 8 is a further embodiment of the invention.

### DESCRIPTION OF THE PREFERRED EMBODIMENT

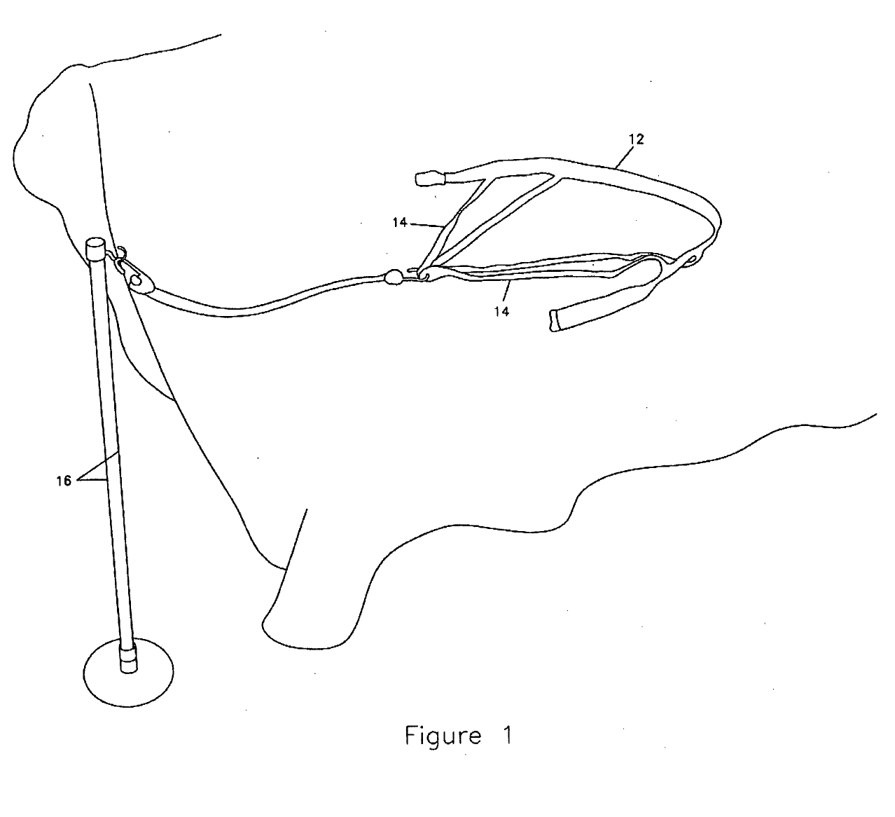

The present invention is described with reference to the enclosed figures wherein the same numbers are utilized. Referring to FIG. 1, the invention, in a first embodiment, is an apparatus for eliminating back tension. In a first embodiment, the invention comprises a waist harness 12 which attaches to an end user 18. Preferably, the user is in a reclining or lying position on a substantially flat surface such as a bed or mat. The harness 12 fits around the waist. The present invention envisions a plurality of attachment mechanisms, including Velcro, hooks, clips or snaps.

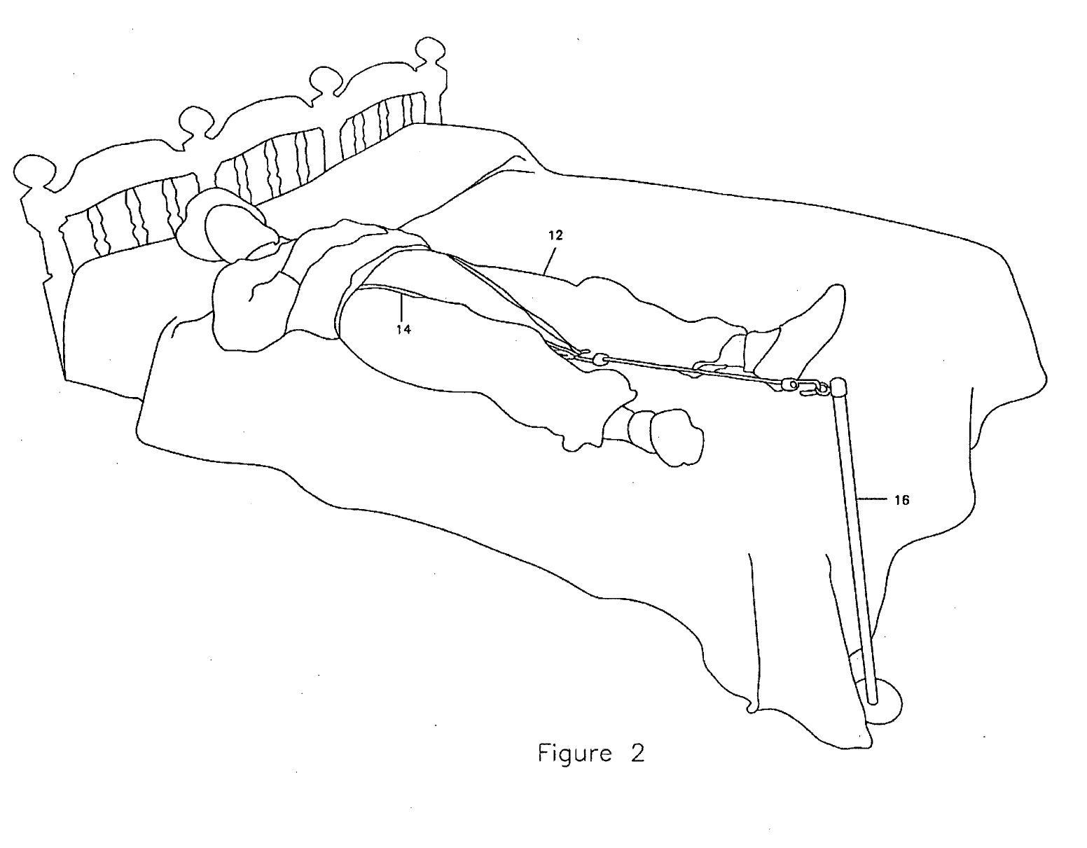

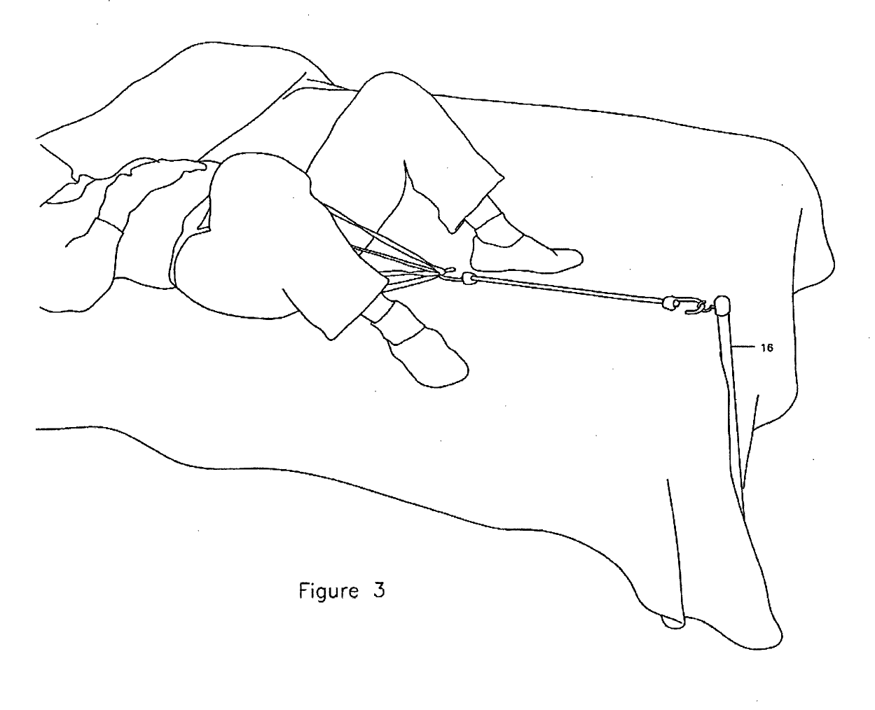

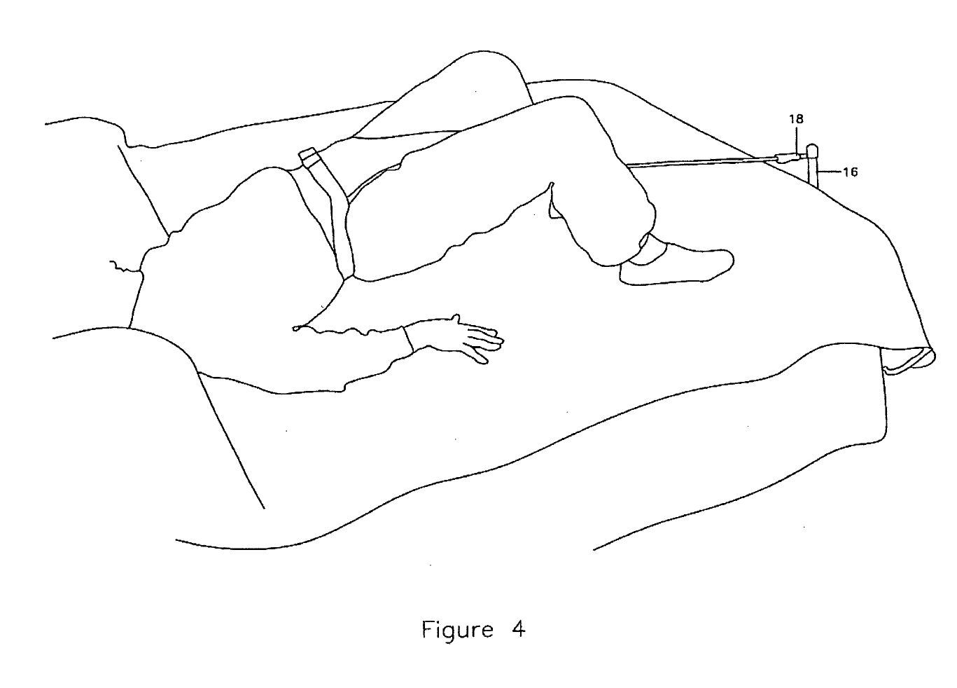

As shown in FIGS. 2 to 5, two strap pairs 14 extend axially down and through the legs of the user 13. The strap pairs attach to a support 16. The system strap pairs 14 and harness 12 place positive pressure on the back of the user when affixed to the support.

In a most preferred embodiment, the straps 14 are affixed by a connector 18 to a variable height pole 16 which is located at an axial location relative to the end user. The attachment of the straps to the pole permits constant pressure to be applied to the end user's back. The height of the pole 16 may be adjustable, based upon the level of the end user and the level of the platform, bed, mat or mattress.

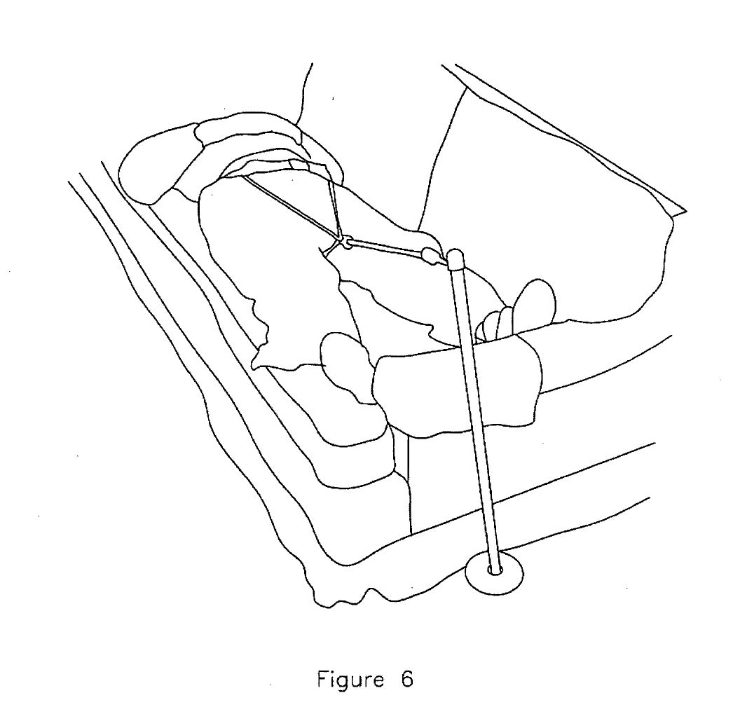

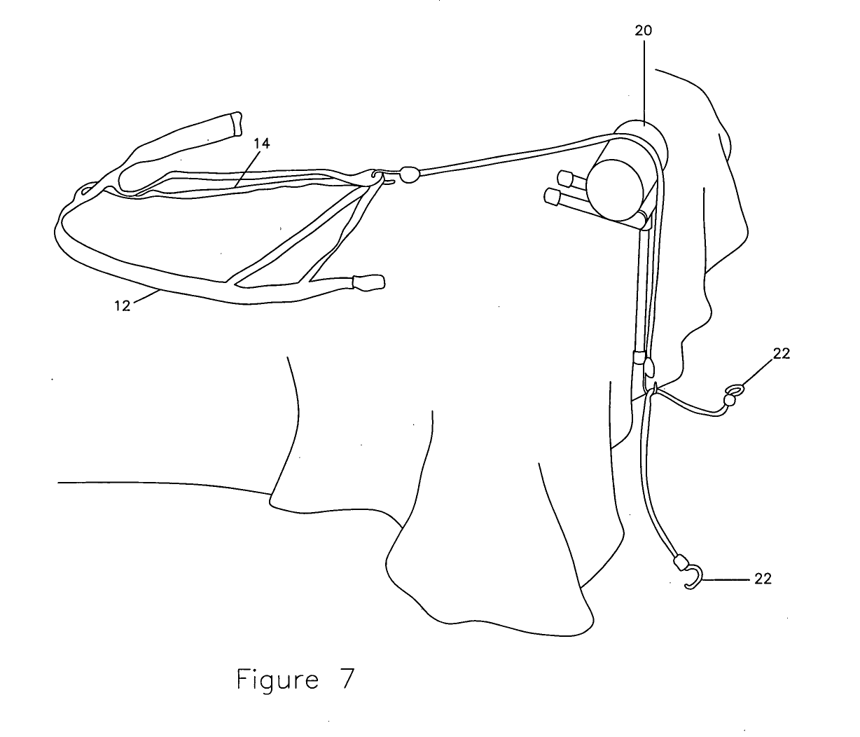

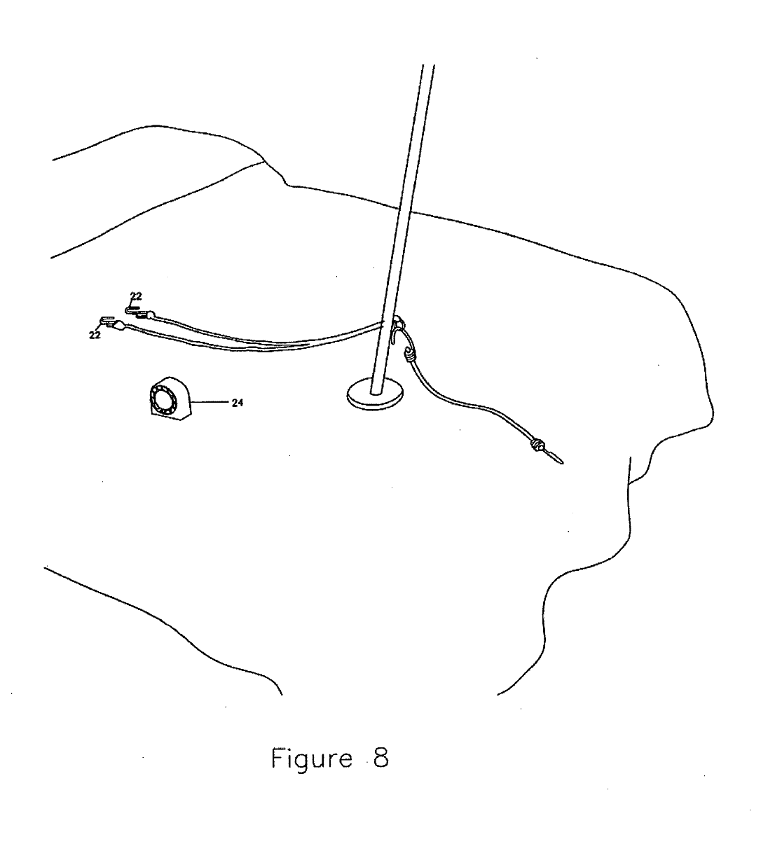

As shown in FIG. 6, in a second embodiment, the strap pairs 14 are secured by the bed and supported upwardly by a pillow or a buttress 20 at the foot of the bed or mattress. A plurality of hooks 22, attach to the carpet or bed frame and secure the straps.

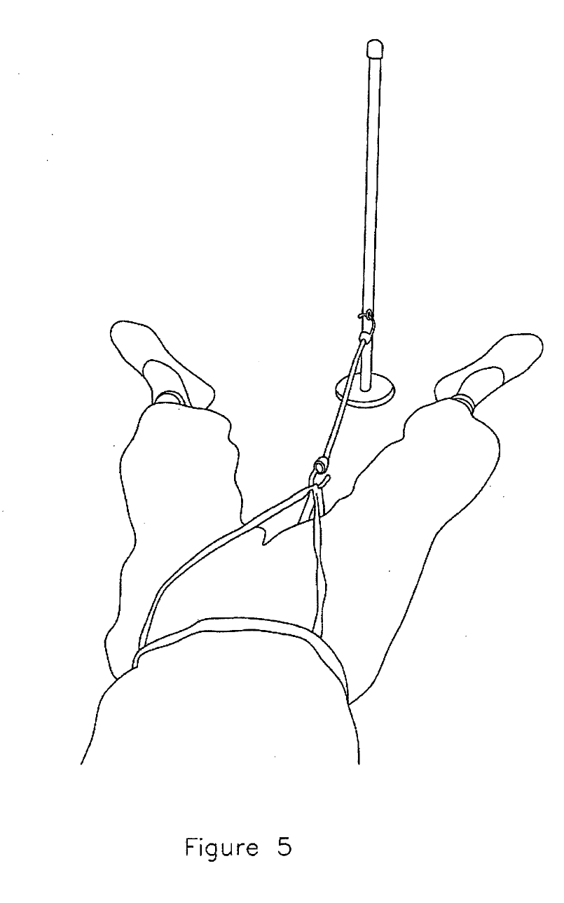

As shown in FIGS. 5 and 6, the operation of the invention is now described. Initially, the waist band is placed on the user in a lying position where the user does not feel any compression lying on his or her back.

While lying on the back with knees up and the feet on the couch (bed or mat) and buttocks lifted, the user pulls toward the head of the bed or mat until slight pressure is placed on the waist belt.

The buttocks of the user are then lowered. If no pressure is felt on the waist of the user, the body should preferably be pulled toward the head of the bed as mild pressure is then gradually applied. The heavier upper torso will hold the belt, letting the lighter bottom torso receive and respond to the light pressure, thus slightly disengaging the vertebrae.

The user can then take different body positions and use the position most comfortable. Body and leg positions can be changed up to 360 degrees at any time, thus slightly changing the pressure points.

A further feature of the invention is the inclusion of a timer. The timer should be used to time usage of the system for between fifteen and thirty minutes. After each usage, the invention should be uncoupled.

The invention should be used in slightly longer decompression periods over time, repeating the decompression and rest periods up to any point where pain or discomfort is felt, but no longer than one hour. These periods could be shorter and used when lying down to watch TV or going to bed and repeated when arising in the morning or other times of your choice. Because the axis of tension is raised above the lying area, the legs can freely rotate and be extended or withdrawn.

The present invention has been described with reference to the enclosed preferred embodiment. It is to be appreciated that the true nature and scope of the present invention is to be determined with reference to the claims appended hereto.

1. A system for relieving back pressure comprising:
a waist belt affixed to a reclining individual;
a plurality of tension lines extending axial through the legs of the individual; and
a mechanism to attach the tension lines so as to provide positive tension.

2. A system for relieving back pressure comprising:
a waist belt affixed to a reclining individual;
a plurality of expandable tension lines extending axially through the legs of the reclining individual and which terminate with a catch; and
an adjustable pole affixed to the catch for adjusting the height of the catch end for providing constant pressure on expandable tension lines and a timer mechanism.

3. A system for relieving back pressure comprising:
an adjustable waist belt affixed to a reclining individual;
a plurality of expandable tension lines extending axially through the legs of the reclining individual and which end with a catch; and
an adjustable pole for affixing the height of the catch end for providing constant pressure on the back of the reclining individual;
and a means coupled to the pole for adjusting the tension of the expandable tension lines.

4. A method for relieving back pressure comprising:
applying a waist belt affixed to a reclining individual;
attaching a plurality of expandable tension lines extending axially
through the legs of the user and which end with a catch; and
affixing the catch to an adjustable pole for affixing the height of the catch end; and shifting the body of the end user to apply continuous or changing pressure on the body.
